(19) United States
(12) Patent Application Publication
(10) Pub. No.: US 2021/0306244 A1
(43) Pub. Date: Sep. 30, 2021

(54) SIGNAL LATENCY DETECTION SYSTEM

(71) Applicant: INFINITE ARTHROSCOPY, INC. LIMITED, Cleveland, OH (US)

(72) Inventors: Ilya MALINSKIY, Cleveland, OH (US); Eugene MALINSKIY, Cleveland, OH (US); Daniel Joseph DUDLEY, Cleveland, OH (US); Patrick Joseph POLITO, Cleveland, OH (US); Daniel Alan NELSON, Cleveland, OH (US); Laimis Kazimieras BELZINSKAS, Cleveland, OH (US); James A. AMOS, Cleveland, OH (US)

(21) Appl. No.: 17/214,493

(22) Filed: Mar. 26, 2021

Related U.S. Application Data
(60) Provisional application No. 62/994,990, filed on Mar. 26, 2020.

(51) Int. Cl. H04L 12/26

(52) U.S. Cl. CPC H04L 43/0858; H04L 43/067; H04L 43/10

(57) ABSTRACT

A latency measurement system configured to measure the latency of an evaluated device, including a reception component, a processing component and an output component, the latency measurement system including an emitter configured to emit a probe signal receivable by a reception component of the evaluated device and processable by a processing component of the evaluated device generating an output signal at an output component of the evaluated device; a processor configured to record a emission time when the probe signal is emitted by the emitter for reception by the processing component of the evaluated device; a detector configured to receive the output signal from the output component of the evaluated device; the processor configured to record a receive time when the detector receives the output signal from the output component of the evaluated device, and calculate a difference between the emission time and the receive time.

### CLAIM OF PRIORITY

This application claims priority to U.S. Provisional Patent Application Ser. No. 62/994,990 filed on Mar. 26, 2020, the contents of which are incorporated herein by reference in their entirety.

### FIELD OF THE INVENTION

The present invention generally relates to measurement systems and methods and, more particularly, to a system and method for measuring latency of devices.

### BACKGROUND

Many systems operate with a latency between an event trigger and a perception of the event that may result from a time to process the event trigger before generating a perceptible output event. In many cases, the latency may have detrimental results particularly in cases where a human action or some important expectation may be dependent on an equivalence between the perception of the event and the event trigger.

One example of a latency-sensitive system involves the use of video camera systems in surgical procedures, including surgical robotic operations. Video camera systems enable clinicians to see their work in the patient using less invasive procedures, which lead to faster recovery times for patients. However, due to video processing and display imaging delays in the camera system itself, clinicians may experience a significant time lag while using the cameras. This processing delay can vary greatly from device to device. However, accurate data depicting latency in these systems is lacking. One difficulty arising from this latency is that the event trigger and the perception of the event correspond with a clinician's sense of vision, which clinicians typically perceive with substantially no delay when cameras are not involved. Too often, a physician may need to rely on sensing a delay through the video system based on the physician's experience with the system and too often, the physician may need to resort to estimating, which is less desirable than a more accurate measurement. In robotic surgical procedures, it is desirable that the time delay between the instruction issued by the surgeon and the movement of the robot responding to the instruction be kept to a minimum. This requires meticulous and accurate measurement and control of latency in such systems. With the increasing popularity and need of telesurgery, telemonitoring, and telementoring, such latency measurements are critical to success of such surgical procedures.

From a practical perspective, the time delay may be potentially dangerous as the physician may be performing an operation during which rapid time response is crucial. If latency is not accurately captured, the chance for errors may increase. Latency may be due to various causes including processing involved, transmission of electromagnetic signals over different paths, wires, and the like. Medical experts assert that the time delay between the perform-time and display-time should be under 100 milliseconds such that a surgeon, for example, is not delayed in their response in a medical procedure. As noted above, added latency by a system may result in a physician needing to guess as to the delay.

Signal processing latency is a problem that is not limited to medical procedures and may include any process when a rapid time response is essential. Other systems, for example, include the drone controllers, arial systems, audio systems, temperature control systems, and any other system in which a human response may be required after no substantial time delay between an event and a perception of the event.

### SUMMARY

In view of the above, examples of latency measurement systems are provided for measuring the latency of an evaluated device that includes a reception component, a processing component and a output component.

In one aspect, an example latency measurement system comprises an emitter configured to emit a probe signal. The emitted probe signal is receivable by the reception component of the evaluated device for processing by the processing component of the evaluated device to generate an output signal at the output component of the evaluated device. The latency measurement system comprises a detector configured to receive the output signal from the output component of the evaluated device. A processor is configured to determine a time delay between the emission of the probe signal by the emitter and receipt of the output signal by the detector.

In one example, the processor is configured to determine the time delay by recording a transmit time when the probe signal is emitted by the emitter for reception by the processing component of the evaluated device. A receive time is then recorded when the detector receives the output signal from the output component of the evaluated device. A difference is calculated between the transmit time when the emitter emits the probe signal to the evaluated device and the receive time when the detector receives the output signal from the evaluated device.

In one example, the processor is configured to determine the time delay by initiating a timer when the probe signal is emitted by the emitter. The timer is read when the output signal is received from the output component of the evaluated device. A timer value is determined from the reading of the timer to be the time delay.

In some examples, the detector includes a light sensor, and the processor is configured to sense a change in brightness at the light sensor indicating a first signal processed by the evaluated device, where the first signal corresponds to a latency in subsequent signals processed by the evaluated device.

In some examples, the emitter is configured to emit a signal having characteristics corresponding to any of the frequencies across the electromagnetic spectrum.

In some examples, the emitter is configured to emit an audio signal, an x-ray signal, an ultraviolet light signal, a visible light signal, an infrared signal, a microwave signal or radio wave signal.

In some examples, the detector is configured to receive an audio signal, an x-ray signal, an ultraviolet light signal, a visible light signal, an infrared signal, a microwave signal or radio wave signal.

In some examples, the reception component is configured to receive an audio signal, an x-ray signal, an ultraviolet light signal, a visible light signal, an infrared signal, a microwave signal or radio wave signal.

In some examples, the output component includes at least one of a video display device or an audio output device.

In some examples, the processor, emitter and detector are disposed within a housing.

In another aspect, a method is provided for measuring a latency in an evaluated device having a reception component, a processing component, and an output component. In an example method, a probe signal is emitted for reception by the reception component of the evaluated device for processing by the processing component of the evaluated device to generate an output signal at the output component of the evaluated device. The output signal is received from the output component of the evaluated device. A time delay between the emission of the probe signal and receipt of the output signal is determined as the latency in the evaluated device.

In one example, the time delay is determined by recording a transmit time when the probe signal is emitted for reception by the processing component of the evaluated device. A receive time is recorded when the output signal is received from the output component of the evaluated device. A difference is calculated between the transmit time of the probe signal to the evaluated device and the receive time when the detector receives the output signal from the evaluated device.

In another example, the time delay is determined by initiating a timer when the probe signal is emitted. The timer is read when the output signal is received from the output component of the evaluated device. A timer value from the reading of the timer is determined to be the time delay of the system.

### BRIEF DESCRIPTION OF THE DRAWINGS

The foregoing summary, as well as the following description of examples of the latency measurement system, will be better understood when read in conjunction with the appended drawings. It should be understood, however, that the example implementations are described below without limiting the scope to the precise arrangements and instrumentalities shown. In the drawings:

FIG. 1 is an example implementation of a latency measurement system.

FIG. 2 is another example implementation of a latency measurement system.

FIG. 3 is another example implementation of a latency measurement system.

FIG. 4 is a flowchart illustrating operation of an example of a method for measuring latency in an evaluated device.

### DETAILED DESCRIPTION OF THE INVENTION

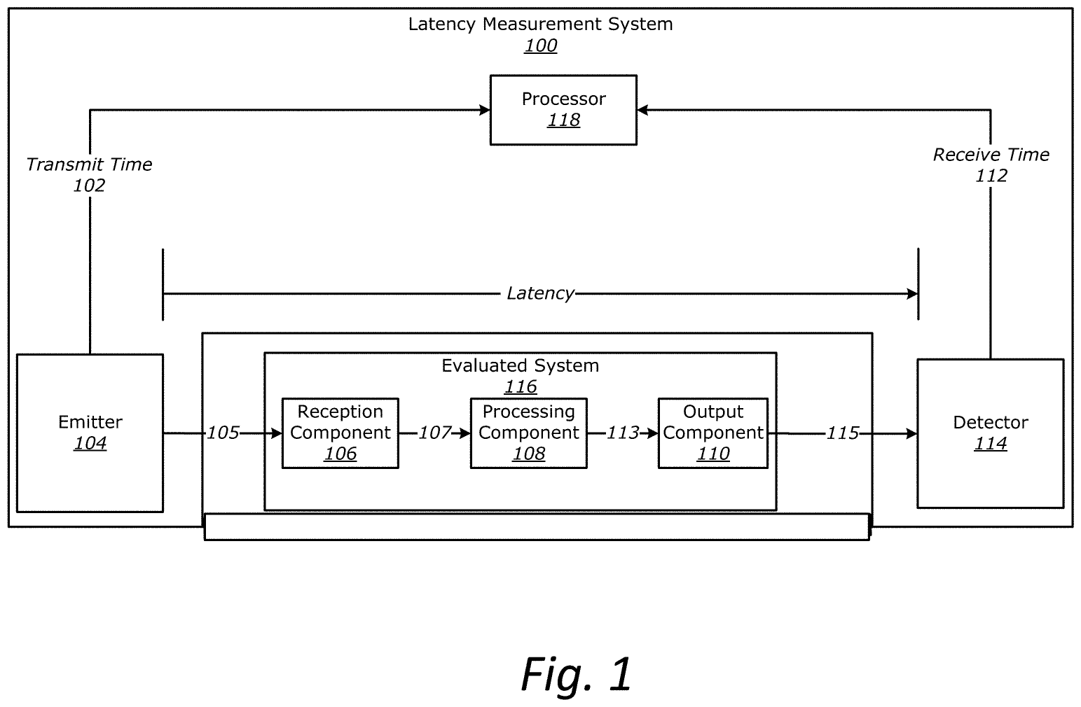

Described below with reference to FIGS. 1-4 are examples of latency measurement systems and methods for measuring a latency in evaluated devices having a reception component, a processing component, and an output component. The evaluated devices may include any system where signals received at the reception component represent a triggering of an event and output signals generated at the output component represent a perception of the triggering of the event. A time delay between the receipt of the signals at the reception component and the generation of output signals at the output component indicate a latency imposed by a processing of the signals received at the reception component in generating the output signals at the output component.

In one example evaluated device, a camera operates as the reception component to receive optical signals, which are then processed by a video processing system to generate an image as an output signal at a display as an output component. In another example, a microphone or audio pickup operates as the reception component to receive audio signals, which are then processed by a sound or audio processing system to generate sound as an output signal at a speaker or speaker system as an output component.

In an example latency measurement system used to determine a latency in a video system, a light emitting device (e.g., light emitting diode, or LED, or LED array, or the like) may operate as an emitter to emit a light as a probe signal for detection by, for example, a camera in the video system. The video system may be, for example, an image system used for performing surgery such that actions by a physician may be picked up by the camera in the video system for presentation on a display screen. The display screen allows the physician to view his or her own actions during, for example, a surgery where the physician would otherwise be unable to see what he or she is doing. The probe signal is processed by the video processing system in a manner similar to the processing of an image or other light patterns by the video processing system. In the meantime, the emission of the probe signal may be detected or noted by a processor of the latency measurement system in order to determine a time delay. The processor may then begin determining the time delay between emission of the probe signal and detection of the output signal by the detector of the latency measurement system. The video processing system in the evaluated device generates an output signal, for example, for display on a display screen. The detector of the latency measurement system may be a light detecting element (e.g., photodiode) configured to detect light from the video system display screen. The processor of the latency measurement system may detect when the detector detects or senses the output signal of the video processing system. In this example, the emission of the light as the probe signal may indicate an event trigger that a human may perceive substantially immediately, but after a time delay when perceived via the display screen due to the latency imposed by the video processing system.

In an example implementation, a video processing system may be configured as the evaluated device to emit a probe signal during an initialization or configuration process before any images are collected by the video processing system. The latency measurement system may then receive indication of the receipt of the output signal from the video processing system and identify the time delay between probe signal emission and output signal reception as the latency of the video processing system. The detection of the output signal at the display screen would be the first signal output since initialization/power up of the evaluated device, and accordingly, the first processed signal output from the evaluated device. Subsequently processed images by the video processing system may be processed with the latency determined from the time delay of the first processed signal.

In another example implementation, a latency measurement system may be used to determine a delay in a sound system, such as for example a home audio system. The emitter may be an audio transducer, or speaker, configured to emit a sound. The sound system may include a microphone connected to a sound processing component of the sound system. The sound processing component may generate a sound similar to the probe signal at an audio output of the sound system. The sound output signal may be picked up by a microphone used by the latency measurement system as the detector. The processor of the latency measurement system may determine a time delay between emission of the probe signal and detection of the output sound signal at the latency measurement system microphone. The time delay may then be reported as the sound system's latency.

In one example of the use of the latency measurement system to determine a time delay in a sound system, the probe signal may be emitted at different frequencies to determine if an imbalance exists in the latency of sounds at different frequencies. Such latency would detrimentally affect the quality of any sound reproduction by the sound system.

In other example implementations, the emitter and detector may be selected according to a specific modality of signals being processed by the evaluated device. In the above briefly described examples, the emitters and detectors used were selected to emit and detect light or images and sound. Other example implementations may be used to determine latencies in processing smell, pressure, temperature, and other modalities. In addition to video processing systems and sound systems, examples of evaluated devices may include ultrasound imaging systems, temperature measurement systems, air pressure systems, electrical systems paired with oscilloscopes, and other systems where a latency between an event and a human's perception of the event after the processing of a sensory modality should be as short as possible.

The reporting of time delays between emission of the probe signal and the detection of the output signal by the evaluated device may be performed in different ways. Where the evaluated device is a video processing system, for example, the latency determined by the latency measurement system may be provided or presented on the display being used by the evaluated device, or by some other visual indicator. In other example implementations, the latency may be simply recorded as a performance specification that may be updated at selected time intervals. In some examples, the measured latency may be used to warn or stop operation of the evaluated device if the latency is determined to pose a danger, such as for example in a surgical setting.

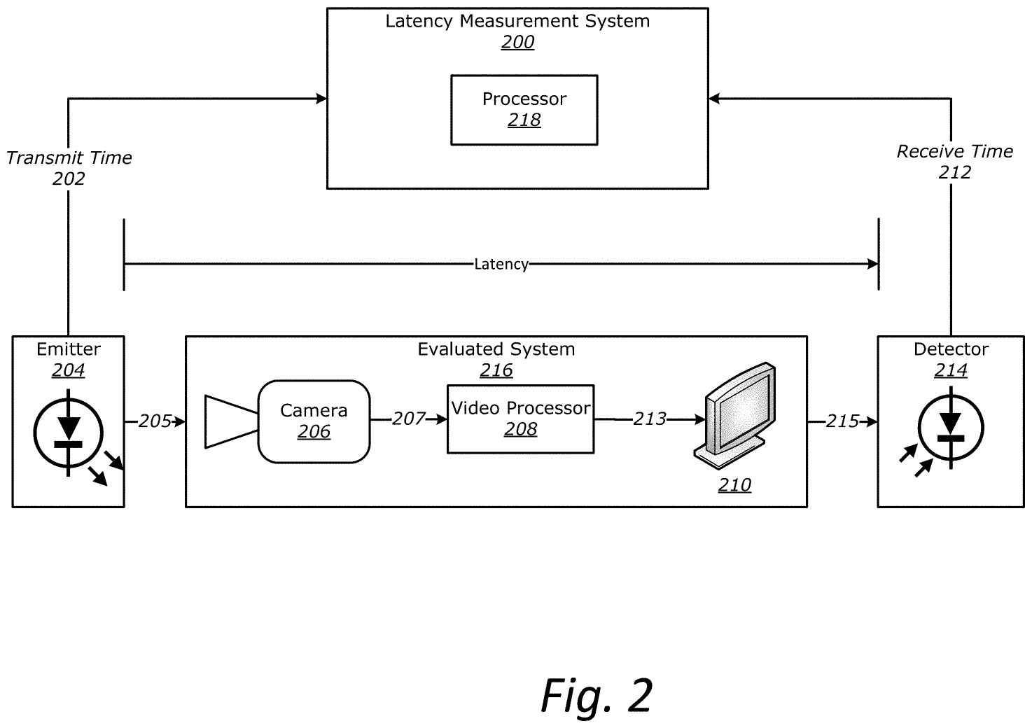

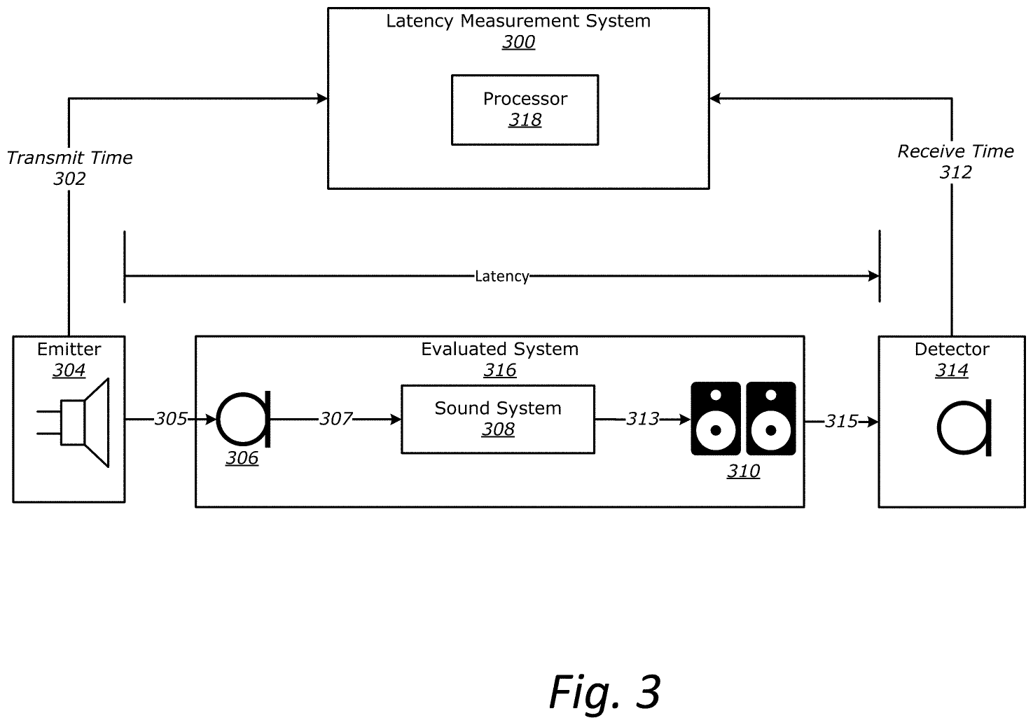

Examples of latency measurement systems and the evaluated devices or systems with which the latency measurement systems may be implemented are described below with reference to FIGS. 1-4. FIG. 1 is a schematic block diagram of an example of a latency measurement system 100 for determining a latency incurred by an evaluated device 116. FIGS. 2 and 3 are schematic block diagrams of examples of the latency measurement system in FIG. 1 for determining latency in the processing of imaging and sound or audio, respectively, by the evaluated devices.

Referring to FIG. 1, a latency measurement system 100 may be configured to measure the latency of an evaluated device 116. The evaluated device 116 may be any device that comprises a reception component 106, a processing component 108 and/or an output component 110. In FIG. 1, the evaluated device 116 is depicted as representing any suitable evaluated device or system. As described below with reference to FIG. 2, the evaluated device may be a video camera system having a camera 206 as a reception component 106 and a display screen 210 as an output component 110. As described below with reference to FIG. 3, the evaluated device may be a sound system having a microphone 306 as the reception component 106 and a speaker 310 as an output component 110. However, example implementations are not limited to use with systems that process either images or sound. In some embodiments, the reception component 106 may be any device configured to receive an electromagnetic signal, such as for example, an x-ray signal, an ultraviolet light signal, a visible light signal, an infrared signal, a microwave signal or radio wave signal. The reception component 106 may be any device configured to detect signals of any suitable modality, including for example, smell, pressure, and temperature. Similarly, in other embodiments, the evaluated system may be a different device or system having different output components. Such devices or systems include for example, an ultrasound system, temperature measuring system, air pressure system, electrical system paired to an oscilloscope, and similar types of systems sensitive to signal or data latency.

As used herein, latency shall be understood to refer to a delay in time between a triggering of an event, such as for example a capture of an image or a sound by an evaluated device, and the perception of the event by a human via a display screen, a speaker, or other output component, where the delay in time is incurred by the processing and/or transferring of data between the reception component and the output component of the evaluated device. Example implementations of the latency measurement system are configured to measure the time delay between the emission of a probe signal to be received by the evaluated device and the detection of the output signal from the output component, and to report the time delay as the latency.

For a video camera system used for surgery, for example, knowing the latency of the video camera system is important because the latency between when the camera sensor receives an image and when the image displayed on the screen can affect how the surgeon performs the surgery. If the latency is too long, from a practical perspective, it could be dangerous for the patient because the surgeon is moving tools, cutting and/or ablating inside the body and the surgeon's reaction time may be compromised by sub-optimal latency of the video camera system.

Referring back to FIG. 1, the latency measurement system 100 may be comprised of an emitter 104, a detector 114 and a processor 118. The emitter 104 of the latency measurement system 100 may be configured to emit a probe signal 105 at a transmit time 102 towards an evaluated system or device 116. The emitter 104 may be any device configured to emit a signal having characteristics corresponding to the frequencies across the electromagnetic spectrum. In one embodiment, the emitter 104 may emit an audio signal, an x-ray signal, an ultraviolet light signal, a visible light signal, an infrared signal, a microwave signal radio wave signal, air pressure, a temperature change, or an aerosol. In other embodiments, the probe signal 105 may comprise other signal modalities such as sound, pressure, smell, temperature, pressure and others. Examples of the reception component 106 may include a camera, an x-ray detector, an antenna, a microphone, an ultrasound sensor, an air pressure sensor, a temperature sensor, or a similar sensor or receiver.

The processor 118 of the latency measurement system 100 may record a transmit time 102 when the probe signal 105 is transmitted by the emitter 104 for reception by the reception component 106 of the evaluated device 116. The recorded transmit time 102 may be used by the processor 118 to calculate a latency of the evaluated device 116, as explained in more detail below. The probe signal 105 may be receivable by a reception component 106 of the evaluated device 116.

In an example implementation, the processor 118 controls the emission of the probe signal 105 by the emitter 104 and may be configured to record the transmit time 102 when the processor 118 controls the emitter 104 to emit the probe signal 105 using the processor system timer. The processor 118 may then record the receive time 112 when the detector 114 detects the output signal 115 from the output component 110 of the evaluated device 116. In other example implementations, the processor 118 may include a timer (not shown) in software or hardware that allows the processor 118 to measure the time delay by starting the timer when the probe signal 105 is emitted and stopping the timer when the detector 114 detects the output signal. The time can then be read to determine the elapsed time as the time delay or latency of the evaluated device 116.

As noted above, the reception component 106 may be any device configured to receive an audio signal, an x-ray signal, an ultraviolet light signal, a visible light signal, an infrared signal, a microwave signal, radio wave signal, or signals of other modalities such as for example, smell, temperature, pressure, and the like. Upon receiving the probe signal 105, the reception component 106 may process the probe signal 105 to generate a reception transmission signal 107. The processing of the probe signal 105 by the reception component 106 may incur additional latency, as it generally takes time to convert a signal from an initial form to another form. It is noted that the latency incurred by transmission of data is a relevant part of the overall latency that the system 100 is configured to measure. The reception transmission signal 107 may be an electrical signal for transmission over a communication path. Examples of communication paths include a wireless communication path and a wired communication path.

The reception component 106 may transmit the reception transmission signal 107 to the processing component 108 of the evaluated device 116 over a communication path. The transmission of the reception transmission signal 107 by the reception component 106 may incur additional latency, as it generally takes time to transmit a signal from one point to another point.

The processing component 108 may process the reception transmission signal 107, resulting in a processed probe signal 113. Examples of processing may include video processing, audio processing, signal transformers, signal storage, digital signal processing, or analytical processing. The processing of the reception transmission signal 107 may incur additional latency, as it generally takes time to convert a signal from one form to another form. Again, the additional latency incurred by the processing component 108 is also a relevant portion of the latency being measured by the system 100.

The processing component 108 may transmit the processed probe signal 113 to the output component 110 of the evaluated device 116 over a communication path. Examples of communication paths include a wireless communication path and a wired communication path. The transmission of the processed probe signal 113 by the processing component 108 may incur additional latency, as it generally takes time to transmit a signal from one point to another point.

The processed probe signal 113 may be processed by the output component 110 of the evaluated device 116 to generate an output signal 115. The output component 110 may then output the output signal 115. The output component 110 may be a video display device, or an audio output device. Other examples of the output component may include a component configured to output an x-ray signal, an ultraviolet light signal, a visible light signal, an infrared signal, a microwave signal a radio wave signal, change in air pressure, a change in temperature, or an aerosol mist. In some embodiments, the output component 110 outputs the output signal 115 as an image displayed on a display screen. In some embodiments, such as for example, the example implementation described below with reference to FIG. 3, the output component 110 outputs the output signal 115 as an audio signal output from speakers of the output component 110. The processing of the processed probe signal 113 by the output component 110 to generate an output signal 115 and then the further presentation of the output signal 115 (e.g., as an image or sound) may incur additional latency, as it generally takes time to convert a signal from one form to another form.

The detector 114 of the latency measurement system 100 may be configured to receive the output signal 115 from the output component 110 of the evaluation device 116 at a receive time 112. The detector 114 may be any device configured to receive an audio signal, an x-ray signal, an ultraviolet light signal, a visible light signal, an infrared signal, a microwave signal, a radio wave signal or similar detectable signal or combination of such signals. In one embodiment, if the output signal 115 is an image, the detector 114 is a photodiode or photodiode array. In one embodiment, if the output signal 115 is sound, the detector 114 is a microphone. In one embodiment, the detector 114 may detect a change in the output signal 115 from the output component 110 at a receive time 112. In some embodiments, the change in the output signal 115 may be a change in brightness. In some embodiments, the change in the output signal 115 may be a change in sound. In other embodiments, the change in output signal 115 may depend on the specific modality or combination of modalities being detected.

The processor 118 may record the receive time 112 when the detector 114 receives the output signal 115 from the output component 110 of the evaluation device 116. The recorded receive time 112 may be used by the processor 118 to calculate a latency of the evaluated device 116, as explained in more detail below.

The processor 118 may calculate a latency of the evaluated device 116 by calculating a difference between the transmit time 102 when the emitter 104 emitted the probe signal 105 and the receive time 112 when the detector 114 received the output signal 115 from the output component 110. The difference between the emission time 102 and the receive time 112 represents the latency of the evaluated device 116. For example, consider the emitter 104 as a light source, the detector 114 as a photodiode and the evaluated device 116 as a video processing system. The difference between when the emitter 104 first emits light to the time when the photodiode detects light from the video processing system is the time that the video processing system used to receive the light from the emitter 104 via a camera, process a signal representative of the light and output a processed signal at a display of the video processing system. By knowing and understanding this information, engineers can design their systems to minimize the system's latency or have more precise latency information, including that the latency is expected by the user of the system.

The latency value obtained from this measurement can then be used to evaluate if the amount of latency is acceptable for the application. In certain circumstances where a high latency is not detrimental to the task that needs to be accomplished, higher latency components may be utilized if they provide cost or component complexity savings. Conversely, if the measured latency of the application in question is deemed to be too high, then the herein proposed latency measuring system can aid in pinpointing the location in the tested application where the latency is the largest and can be improved upon in the most effective way.

As shown in FIG. 1, the latency measurement system 100 may encompass a complete system including the processor 118, emitter 104, and detector 114. The processor 118, emitter 104 and detector 114 may be disposed within a housing. Disposing processor 118, emitter 104 and detector 114 may help to protect the components from damage from use or exposure to external environmental factors (e.g., wind, rain).

In a second embodiment of the latency measurement system 100, the emitter 104 and detector 114 may be disposed in separate housings and include a method for accurately synchronizing a timer in the each of the emitter 104 and detector 114. Such an embodiment would allow for the measuring of applications where a single housing would prove detrimental to measuring the latency of the application, especially when there exists a large distance between the reception component 106 and output component 110. Further, such an embodiment may provide improved accuracy of the latency measurement when the need is extraordinarily high, and a synchronized time provides further increase in accuracy.

Referring to FIG. 2, a latency measurement system 200 is illustrated in an example where the evaluated system or device is a video processing system 216. As shown in FIG. 2, the latency measurement system 200 is configured to measure the latency of the video processing system 216 that includes a camera 206, a video processing component 208, and a display screen 210. The latency measurement system 100 may include an emitter 204 (such as for example a light emitting device, or light emitting diode (LED), an LED array or similar emitting device) configured to emit a probe signal 205 receivable by the camera 206 of the video processing system 216 and processable by the video processing component 208 of the video processing system 216 to generate an electrical output signal 213 representing an image or light signal at the display screen 214. The latency measurement system 200 may also include a processor 218 configured to record a transmit time 202 when the probe signal 205 is emitted by the light or image emitter 204 for reception by the processing component of the video processing system 216. The latency measurement system 200 may also include a light detector 214 (e.g., photodiode, photodiode array, camera or similar detector) configured to receive the output signal from the output component of the video processing system 216. The processor 218 may be configured to record a receive time (e.g., receive time 212) when the detector 214 receives the output signal from the display screen 210 of the video processing system 216. The processor 218 may also calculate a difference between the transmit time 202 when the emitter 204 emits the probe signal 205 to the video processing system 216 and the receive time 212 when the light detector 214 receives the processed signal from the evaluated video processing system 216.

Referring to FIG. 3, a latency measurement system 300 is illustrated in an example where the evaluated system or device is a sound system 316. As shown in FIG. 3, the latency measurement system 300 is configured to measure the latency of the sound system 316 that includes a sound transducer 306 (e.g., microphone, piezoelectric transducer or similar sound receiving device), a sound processing component 308, and an audio output device 310 (e.g., speaker or similar audio output producing device). The latency measurement system 300 may include an audio output device (e.g., speaker or the like) 304, configured to emit a sound probe signal 305 receivable by the sound transducer 306 of the sound system 316 and processable by the sound processing component 308 of the sound system 316 to generate an electrical output signal 313 representing a sound signal output 315 by the sound system speaker 310. The audio output signal 315 is sensed by a sound detector 314 in the latency measurement system 300. The latency measurement system 300 may also include a processor 318 configured to record a transmit time 302 when the audio probe signal 305 is emitted by the audio output device 304 for reception by the processing component of the sound system 316. The latency measurement system 300 may also include the sound detector 314 (e.g., microphone, piezoelectric transducer or similar sound receiving device) configured to receive the audio output signal 315 from the audio output device 310 of the sound system 316. The processor 318 may be configured to record a receive time (e.g., receive time 312) when the detector 314 receives the audio output signal 315 from the audio output device 310 of the sound system 316. The processor 318 may also calculate a difference between the transmit time 302 when the emitter 304 emits the probe signal 305 to the video processing system 316 and the receive time 312 when the detector 314 receives the processed audio output signal 315 from the sound system 316.

In one embodiment, the latency measurement system 100 includes one or more computers having one or more processors and memory (e.g., one or more nonvolatile storage devices). In some embodiments, memory or computer readable storage medium of memory stores programs, modules and data structures, or a subset thereof for a processor to control and run the various systems and methods disclosed herein. In one embodiment, a non-transitory computer readable storage medium having stored thereon computer-executable instructions which, when executed by a processor, perform one or more of the methods disclosed herein.

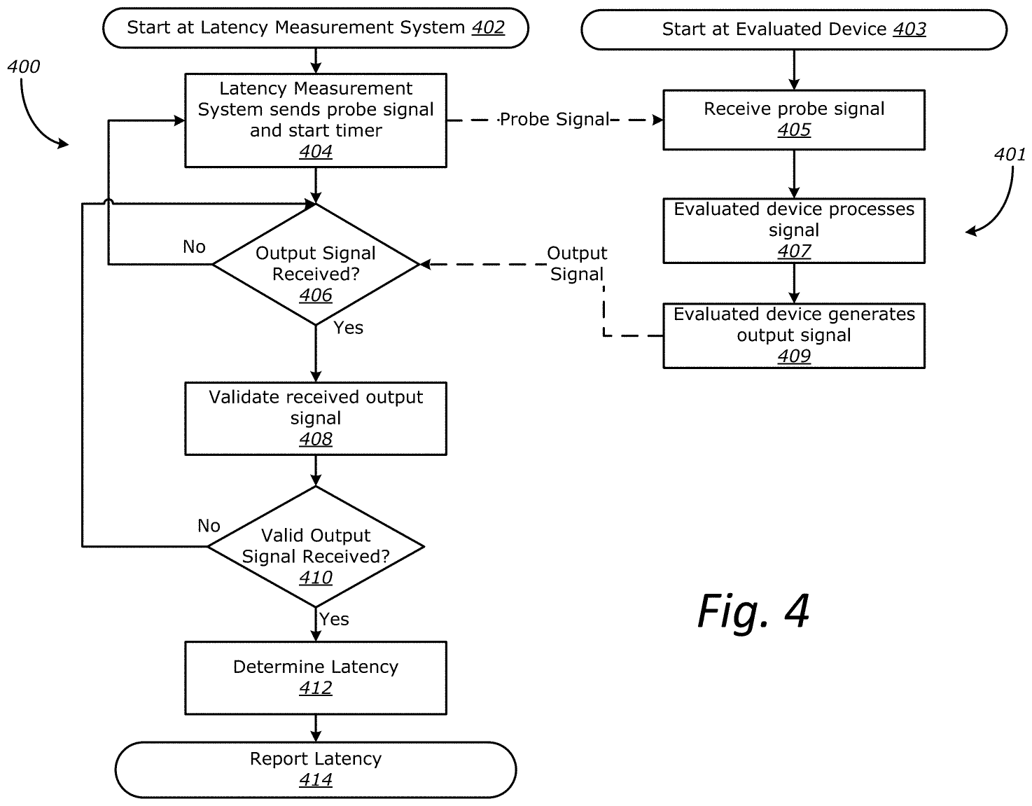

FIG. 4 is a flowchart illustrating operation of an example of a method 400 for measuring latency in an evaluated device. The method 400 of measuring latency is illustrated in parallel with an example method 401 performed by an evaluated device (or system under test) to depict examples of the interaction that may occur in measuring the latency of the evaluated device. In the description of the methods 400 and 401 in FIG. 4, reference is made to components in FIG. 1 unless otherwise indicated below.

The method 400 may start at 402 by emitting a probe signal at step 404 that may be received by the evaluated device at step 405 of the method 401. Also, at step 404, a transmit time is received by the processor 118 to start timing of the latency of the evaluated system 116. A timer may also be started by the processor. Decision block 406 of method 400 represents a waiting for a detection of an output signal from the evaluated device as a continuous check for receipt of the output signal. If an output signal has not been received, the waiting is represented by the “No” path of decision block 406. If an output signal is received at the detector 114 of the latency measurement system 100 (“Yes” path from decision block 406), the received output signal may be validated at step 408 to determine the received output signal from the evaluated device is valid. At step 408, the timer of the processor may be stopped, or a receive time is noted by the processor of the latency measurement system 100.

The validation of the output signal may take many forms depending on specific implementations. In an imaging system as the evaluated system, the validation may involve correlating a pattern in the image with a pattern in the probe signal. In a sound system as the evaluated device, the validation may involve correlating a frequency of the output signal received from the evaluated device with the frequency of the audio probe signal.

The output signal received at step 408 may be output by the evaluated device when the probe signal is received at step 405, then processed at step 407 of the method 401 by the evaluated device. At step 407, the evaluated device generates the output signal 409 for detection by the detector of the latency measurement system.

Decision block 410 of method 400 determines if the output signal received is valid. If the output signal is not valid (“No” path), the method 400 returns to waiting for the latency to end at decision block 406. If the output signal is valid (“Yes” path), the latency is determined at step 412. The latency may be determined from a difference between the receive time when the output signal is received from the evaluated device and the transmit time of the emission of the probe signal. Once determined, the latency may be reported at step 414. The reporting of the latency may be done using a visual medium, such as for example, the display screen used by the video processing system in FIG. 2, or a display device in any evaluated device. The reporting of the latency may be done using an audio message using the audio output device of the sound system 316 in FIG. 3, or an audio output device in any evaluated device. The reporting of the latency may also be recorded as a performance specification that may be monitored.

It will be appreciated by those skilled in the art that latency measuring systems and methods of measuring latency may be configured and may depend on the specific modality or combination of modalities being detected. Specifically, processing signals such as video, sound, light, pressure, special recognition signals and the like over time may be impacted or otherwise affected as they are processed by the brain. Such processing may be considered, approximated, or predicted and may include linear prediction which is a mathematical operation where future values of a discrete spatial, temporal or similar signal are estimated as a linear function of previous samples. Further, such processing may be subject to or enhanced by temporal logic for specifying properties over time, including behavior of a finite-state system. Temporal logic may be employed in various forms including propositional temporal logic, real-time temporal logic, metric temporal logic, signal temporal logic or similar models which may approximate or predict the overall behavior of complex systems.

It will be appreciated by those skilled in the art that changes may be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

I/We claim:

1. A latency measurement system configured to measure the latency of an evaluated device having a reception component, a processing component, and an output component, the latency measurement system comprising:
an emitter configured to emit a probe signal received by the reception component of the evaluated device for processing by the processing component of the evaluated device to generate an output signal at the output component of the evaluated device;
a detector configured to receive the output signal from the output component of the evaluated device; and
a processor configured to determine a time delay between the emission of the probe signal by the emitter and receipt of the output signal by the detector.

2. The latency measurement system of claim 1 where the processor is configured to determine the time delay by:
recording a transmit time when the probe signal is emitted by the emitter for reception by the processing component of the evaluated device;
recording a receive time when the detector receives the output signal from the output component of the evaluated device; and
calculate a difference between the transmit time when the emitter emits the probe signal to the evaluated device and the receive time when the detector receives the output signal from the evaluated device.

3. The latency measurement system of claim 1 where the processor is configured to determine the time delay by:
initiating a timer when the probe signal is emitted by the emitter;
reading the timer when the output signal is received from the output component of the evaluated device; and
determining a timer value from the reading of the timer.

4. The latency measurement system of claim 1, wherein:
the detector includes a light sensor; and
the processor is further configured to sense a change in brightness at the light sensor indicating a first signal processed by the evaluated device.

5. The latency measurement system of claim 1, wherein:
the emitter is configured to emit a signal having characteristics corresponding to any of the frequencies across the electromagnetic spectrum.

6. The latency measurement system of claim 5, wherein the emitter is configured to emit an audio signal, an x-ray signal, an ultraviolet light signal, a visible light signal, an infrared signal, a microwave signal, a radio wave signal, pressurized air, heat, or an aerosol.

7. The latency measurement system of claim 1, wherein the detector is configured to receive an audio signal, an x-ray signal, an ultraviolet light signal, a visible light signal, an infrared signal, a microwave signal, a radio wave signal, an air pressure, a change in temperature, or an aerosol.

8. The latency measurement system of claim 1, wherein the reception component is configured to receive an audio signal, an x-ray signal, an ultraviolet light signal, a visible light signal, an infrared signal, a microwave signal or radio wave signal, an air pressure, a change in temperature, or an aerosol.

9. The latency measurement system of claim 1, wherein the output component includes at least one of a video display device, or an audio output device.

10. The latency measurement system of claim 1, wherein the processor, emitter and detector are disposed within a housing.

11. The latency measurement system of claim 1, wherein the emitter and detector are disposed within separate housings.

12. The latency measurement system of claim 11, further comprising a timer for each of the emitter and the detector, wherein the timer for the emitter and the detector are accurately synchronized.

13. A method of measuring a latency in an evaluated device having a reception component, a processing component, and an output component, the method comprising:
emitting a probe signal receivable by the reception component of the evaluated device for processing by the processing component of the evaluated device to generate an output signal at the output component of the evaluated device;
receiving the output signal from the output component of the evaluated device; and
determining a time delay between the emission of the probe signal and receipt of the output signal.

14. The method of claim 13 where determining the time delay further comprises:
recording a transmit time when the probe signal is emitted for reception by the processing component of the evaluated device;
recording a receive time when the output signal is received from the output component of the evaluated device; and
calculating a difference between the transmit time of the probe signal to the evaluated device and the receive time when the detector receives the output signal from the evaluated device.

15. The method of claim 13 where determining the time delay further comprises:
initiating a timer when the probe signal is emitted;
reading the timer when the output signal is received from the output component of the evaluated device; and
determining a timer value from the reading of the timer.

16. The method of claim 13, where the output signal is a light signal, and the step of receiving the output signal includes sensing a change in brightness indicating a first signal processed by the evaluated device.

17. The method of claim 13, wherein the step of emitting the probe signal includes emitting a light signal having characteristics corresponding to any of the frequencies across the electromagnetic spectrum.

18. The method of claim 17, wherein the step of emitting the probe signal includes emitting an audio signal, an x-ray signal, an ultraviolet light signal, a visible light signal, an infrared signal, a microwave signal, a radio wave signal, pressurized air, heat, or an aerosol.

19. The method of claim 13, wherein the step of receiving the output signal includes receiving an audio signal, an x-ray signal, an ultraviolet light signal, a visible light signal, an infrared signal, a microwave signal, a radio wave signal, an air pressure, a change in temperature, or an aerosol.

20. The method of claim 13 further comprising accurately synchronizing a timer for emitting the probe signal and receiving the output signal.

21. The method of claim 13, wherein the probe signal has a strength, a wavelength, an amplitude, a duration, and a frequency, and wherein strength, wavelength, amplitude, duration, or frequency of the probe signal is configured to be detectable by the reception component of the evaluated device.

22. The method of claim 13, wherein the reception component has a sensitivity that is sufficient to detect the probe signal after traveling through the processing component of the evaluated device.

23. The method of claim 21 further comprising providing a synchronization signal confirming that the evaluated device is in a state amenable to being tested.
